(19) United States
(12) Patent Application Publication
(10) Pub. No.: US 2017/0131563 A1
(43) Pub. Date: May 11, 2017

(54) LENS ASSEMBLY

(71) Applicant: QBAS CO., LTD., Taipei (TW)

(72) Inventor: Chih-Cheng SHIUE, Taipei (TW)

(73) Assignee: QBAS CO., LTD., Taipei (TW)

(21) Appl. No.: 15/265,712

(22) Filed: Sep. 14, 2016

(30) Foreign Application Priority Data
Nov. 11, 2015 (TW) 104137140

(51) Int. Cl. G02C 5/00; G02C 3/00; A61F 9/02; G02C 7/10

(52) U.S. Cl. CPC G02C 5/001; G02C 7/10; G02C 3/003; A61F 9/027; G02C 2200/08; G02C 2200/04

(57) ABSTRACT

A lens assembly that can be quickly released and assembled is provided. The lens assembly has a lens structure, a fixing structure and a quick release structure. The fixing structure can fix the lens structure around the eyes of a user. The quick release structure has a first quick release portion and a second quick release portion. The first quick release portion is disposed on the lens structure, and the second quick release portion is disposed on the fixing structure. The lens structure and the fixing structure can be assembled and disassembled rapidly by means of the first quick release portion and the second quick release portion without additional tools.

### CROSS-REFERENCES TO RELATED APPLICATIONS

This application claims priority to Taiwan Patent Application No. 104137140 filed on Nov. 11, 2015, which is hereby incorporated herein by reference in its entirety.

### BACKGROUND OF THE INVENTION

1. Field of the Invention

The present invention relates to a lens assembly, and more particularly, relates to a lens assembly that can be quickly released and assembled.

2. Descriptions of the Related Art

In the prior art, glasses having only the decorative effect, sunglasses having the sun shading effect, or goggles having the waterproofing effect are all manufactured to have a single function.

For example, the glasses having only the decorative effect cannot provide the UV-blocking or waterproofing function; and similarly, the sunglasses cannot provide the waterproofing effect during water activities, and the goggles worn by a user during a water activity cannot provide the user with the sun shading effect.

Therefore, when a user is in an outdoor sport such as the triathlon, the user has to carry at least a pair of goggles and at least a pair of sunglasses with him/her to meet wearing requirements of different events because he/she needs to finish swimming, cycling and a modified Marathon race in sequence. However, carrying the goggles and the sunglasses often represents a burden for the user.

Accordingly, an urgent need exists in the art to provide a multifunctional lens assembly that can be quickly released and assembled to meet the different wearing requirements such as sun shading or waterproofing.

### SUMMARY OF THE INVENTION

An objective of the present invention is to provide a lens assembly that can be quickly released and assembled according to different wearing requirements so as to achieve the purpose of sun shading or waterproofing.

To achieve the aforesaid objective, a lens assembly that can be quickly released and assembled of the present invention comprises a lens structure, a fixing structure and a quick release structure. The fixing structure is adapted to fix the lens structure around the eyes of a user. The quick release structure has a first quick release portion and a second quick release portion, the first quick release portion is disposed on the lens structure, and the second quick release portion is disposed on the fixing structure. The lens structure and the fixing structure can be assembled and disassembled rapidly by means of the first quick release portion and the second quick release portion without additional tools.

To achieve the aforesaid objective, the lens structure of the lens assembly of the present invention comprises a skirt portion that can cover the face of the user as wearing the lens structure.

To achieve the aforesaid objective, the lens structure of the lens assembly of the present invention is a pair of goggles or a pair of sunglasses, and the fixing structure is a temple-type fixing structure or a belt-type fixing structure.

To achieve the aforesaid objective, the quick release structure of the lens assembly of the present invention is a snap structure or a latch structure.

To achieve the aforesaid objective, the first quick release portion of the lens assembly of the present invention is disposed on the lens structure by being assembled to or integrally formed with the lens structure.

To achieve the aforesaid objective, a lens assembly that can be quickly released and assembled of the present invention comprises a first lens structure, a second lens structure, a fixing structure and a quick release structure. The second lens structure is releasably disposed on the first lens structure. The fixing structure is adapted to fix the first lens structure around the eyes of a user. The quick release structure has a first quick release portion and a second quick release portion, the first quick release portion is disposed on the second lens structure and the second quick release portion is disposed on the fixing structure. The second lens structure and the fixing structure can be assembled and disassembled rapidly by means of the first quick release portion and the second quick release portion without additional tools.

To achieve the aforesaid objective, the second lens structure of the lens assembly of the present invention can be rapidly disposed on the first lens structure or disassembled from the first lens structure without additional tools.

To achieve the aforesaid objective, the first lens structure of the lens assembly of the present invention comprises a skirt portion that can cover the face of the user as wearing the lens structure.

To achieve the aforesaid objective, the first lens structure of the lens assembly of the present invention is a pair of goggles, the second lens structure is a pair of sunglasses, and the quick release structure is a snap structure or a latch structure.

To achieve the aforesaid objective, the first quick release portion of the lens assembly of the present invention is disposed on the second lens structure by being assembled to or integrally formed with the second lens structure.

To achieve the aforesaid objective, a lens assembly that can be quickly released and assembled of the present invention comprises a lens frame, a set of lenses, a fixing structure and a quick release structure. The set of lenses is adapted to be fixed on the lens frame. The fixing structure is adapted to fix the lens frame around the eyes of a user. The quick release structure has a first quick release portion and a second quick release portion, the first quick release portion is disposed on the lens frame, and the second quick release portion is disposed on the fixing structure. The lens frame and the fixing structure can be assembled and disassembled rapidly by means of the first quick release portion and the second quick release portion without additional tools.

To achieve the aforesaid objective, the set of lenses of the lens assembly of the present invention is a pair of goggles or a pair of sunglasses.

To achieve the aforesaid objective, the set of lenses of the lens assembly of the present invention includes two lenses, each of the lenses has a peripheral portion, each of the lenses defines an indentation portion at a place nearby but not contacting the peripheral portion, and a skirt portion is disposed on the indentation portion.

To achieve the aforesaid objective, the fixing structure of the lens assembly of the present invention is a temple-type fixing structure or a belt-type fixing structure, and the quick release structure is a snap structure or a latch structure.

To achieve the aforesaid objective, the first quick release portion of the lens assembly of the present invention is disposed on the lens frame by being assembled to or integrally formed with the lens frame.

The detailed technology and preferred embodiments implemented for the subject invention are described in the following paragraphs accompanying the appended drawings for people skilled in this field to well appreciate the features of the claimed invention.

### BRIEF DESCRIPTION OF THE DRAWINGS

FIG. 1 is a schematic view of a fixing structure as a belt-type fixing structure in a first embodiment of a lens assembly according to the present invention;

FIG. 2 is a schematic view of a fixing structure as a temple-type fixing structure in the first embodiment of the lens assembly according to the present invention;

FIG. 3 is a schematic view of a lens structure comprising a skirt portion in the first embodiment of the lens assembly according to the present invention;

FIG. 4 is a schematic view of a quick release structure in the first embodiment of the lens assembly according to the present invention;

FIG. 5 is a schematic view of a fixing structure as a belt-type fixing structure when the lens structure has the skirt portion in the first embodiment of the lens assembly according to the present invention;

FIG. 6 is a schematic view of a fixing structure as a temple-type fixing structure when the lens structure has the skirt portion in the first embodiment of the lens assembly according to the present invention;

FIG. 7 is a schematic assembled view of FIG. 6;

FIG. 8 is a schematic view illustrating another aspect of a lens structure comprising a skirt portion in the first embodiment of the lens assembly according to the present invention;

FIG. 9 is a schematic view of a first lens structure in a second embodiment of a lens assembly according to the present invention;

FIG. 10 is a schematic view of a second lens structure in the second embodiment of the lens assembly according to the present invention;

FIG. 11 is a schematic view of the second lens structure disposed on the first lens structure in the second embodiment of the lens assembly according to the present invention;

FIG. 12 is a schematic view of a fixing structure as a belt-type fixing structure in the second embodiment of the lens assembly according to the present invention;

FIG. 13 is a schematic view of a fixing structure as a temple-type fixing structure in the second embodiment of the lens assembly according to the present invention;

FIG. 14 is a schematic view of a lens frame in a first aspect of a third embodiment of a lens assembly according to the present invention;

FIG. 15 is a schematic view of a set of lenses in the first aspect of the third embodiment of the lens assembly according to the present invention;

FIG. 16 is a schematic view of the set of lenses assembled with the lens frame in the first aspect of the third embodiment of the lens assembly according to the present invention;

FIG. 17 is a schematic view of a fixing structure as a belt-type fixing structure in the first aspect of the third embodiment of the lens assembly according to the present invention;

FIG. 18 is a schematic view of a fixing structure as a temple-type fixing structure in the first aspect of the third embodiment of the lens assembly according to the present invention;

FIG. 19 is a schematic view of a lens frame in a second aspect of the third embodiment of the lens assembly according to the present invention;

FIG. 20 is a schematic view of a set of lenses in the second aspect of the third embodiment of the lens assembly according to the present invention;

FIG. 21 is a schematic view of the set of lenses assembled with the lens frame in the second aspect of the third embodiment of the lens assembly according to the present invention;

FIG. 22 is a schematic view of a fixing structure as a belt-type fixing structure in the second aspect of the third embodiment of the lens assembly according to the present invention; and

FIG. 23 is a schematic view of a fixing structure as a temple-type fixing structure in the second aspect of the third embodiment of the lens assembly according to the present invention.

### DESCRIPTION OF THE PREFERRED EMBODIMENT

The present invention relates to a lens assembly with structures that can be quickly released and assembled so that the lens assembly can be adjusted to different usage modes according to different wearing requirements of a user.

A first embodiment of a lens assembly of the present invention is described hereinafter.

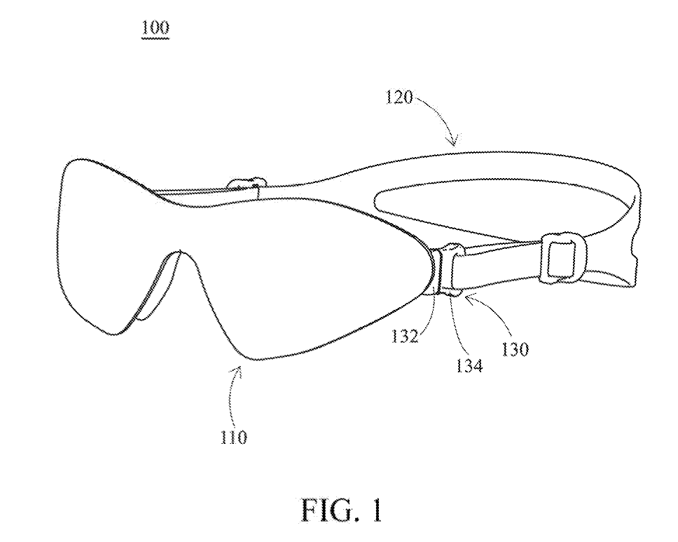

Referring to FIG. 1, in the first embodiment of the present invention, a lens assembly 100 comprises a lens structure 110, a fixing structure 120 and a quick release structure 130. The fixing structure 120 is adapted to fix the lens structure 110 around the eyes of a user (not shown). The quick release structure 130 has a first quick release portion 132 and a second quick release portion 134, the first quick release portion 132 is disposed on the lens structure 110, and the second quick release portion 134 is disposed on the fixing structure 120.

In this way, owing to the arrangement of the first quick release portion 132 and the second quick release portion 134, the lens structure 110 and the fixing structure 120 can be rapidly assembled and disassembled by the user simply through a snap operation without using additional tools (e.g., a screwdriver).

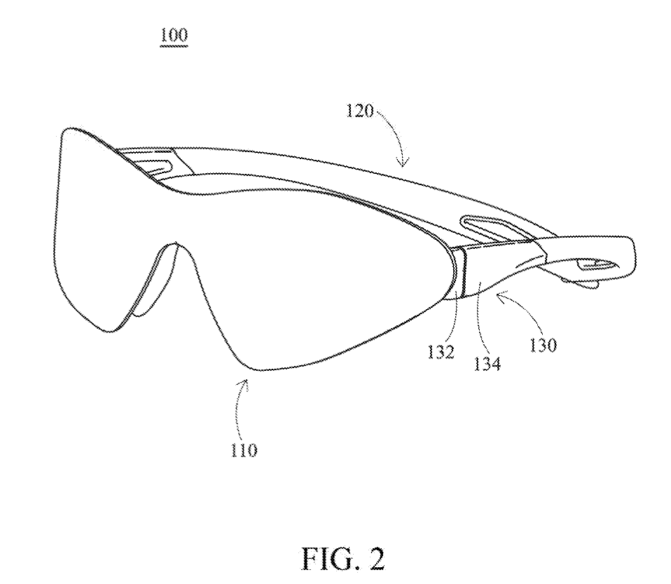

It shall be appreciated that, in the embodiment of FIG. 1, the first quick release portion 132 is disposed on the lens structure 110 by being assembled to or integrally formed with the lens structure 110, and the fixing structure 120 is a belt-type fixing structure for assisting in fixing the lens structure 110 around the eyes of the user to prevent the possible dropping of the lens structure 110 due to strenuous movement. However, as shown in FIG. 2, the fixing structure 120 may also be a temple-type fixing structure so as to provide more comfortable wearing feeling to the user.

In addition to the aforesaid sun shading effect, the lens assembly 100 of the present invention may also have the waterproofing effect by being provided with other elements.

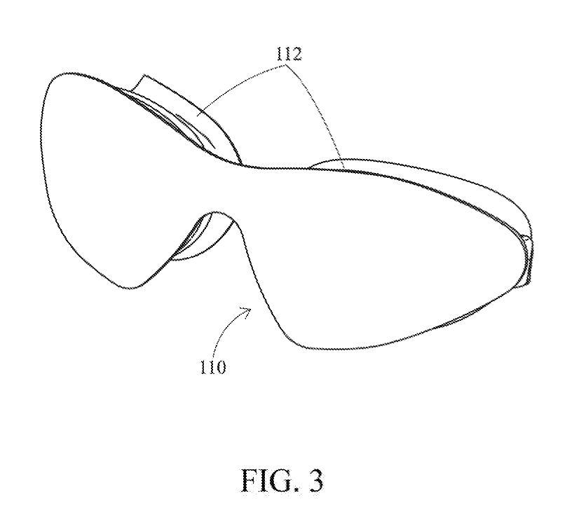

For example, in the embodiment of FIG. 3, the lens structure 110 of the lens assembly 100 may further comprise a skirt portion 112. Therefore, the skirt portion 112 can be used to further cover the face of the user when the lens assembly is worn during a water activity, thereby achieving the waterproofing effect and protecting the eyes.

In this way, the lens structure 110 of the aforesaid lens assembly 100 can be flexibly converted into goggles or sunglasses depending on different usage requirements or different activity places.

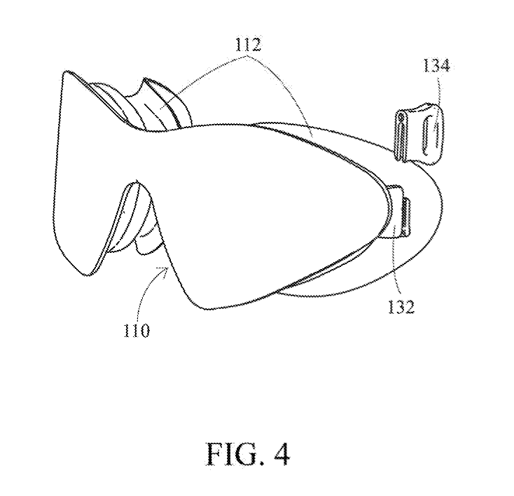

On the other hand, the first quick release portion 132 and the second quick release portion 134 of the quick release structure 130 of the present invention may also have the implementation as shown in FIG. 4 in a preferred embodiment. That is, the first quick release portion 132 and the second quick release portion 134 can be rapidly released and assembled by being snapped vertically. Therefore, the first quick release portion 132 and the second quick release portion 134 of the quick release structure 130 may together form a snap structure or a latch structure.

However, the aforesaid quick release structure 130 is not limited to the forms of a snap structure or a latch structure, and other joining manners such as sleeve coupling may also be possible.

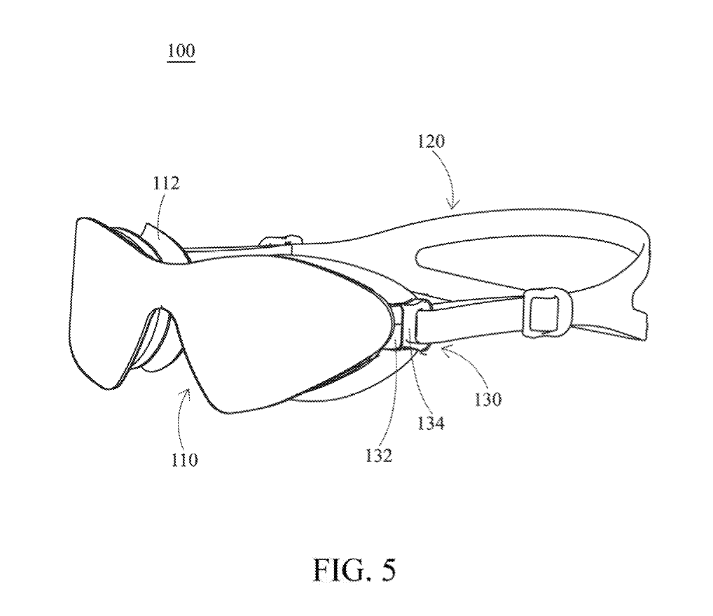

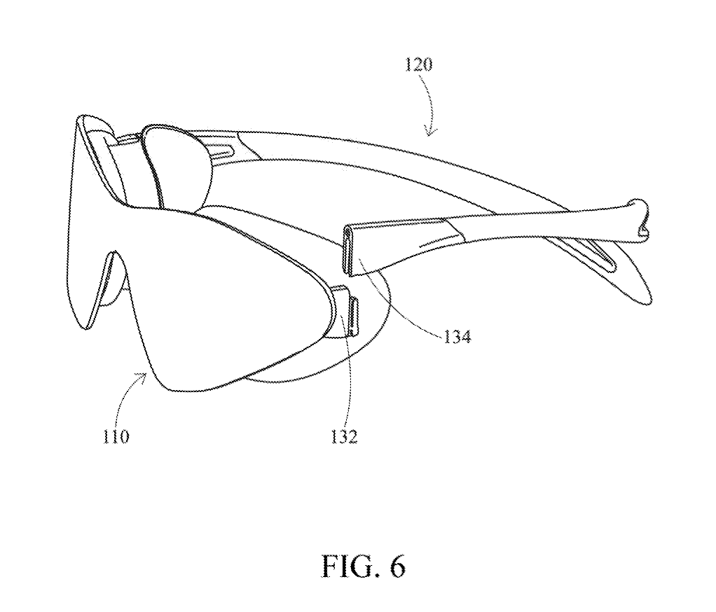

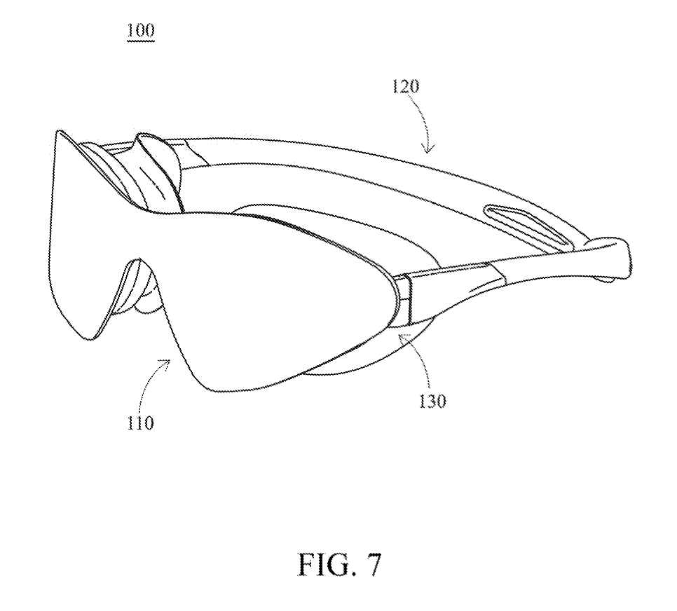

Thereafter, as shown in FIG. 5, the fixing structure 120 in the belt form is bonded with the quick release structure 130 so that the assembling is finished rapidly. Of course, as shown in FIG. 6 and FIG. 7, the fixing structure 120 may also be selected to be a temple-type fixing structure by the user depending on his/her personal preference so that the lens assembly 100 is more comfortable to wear.

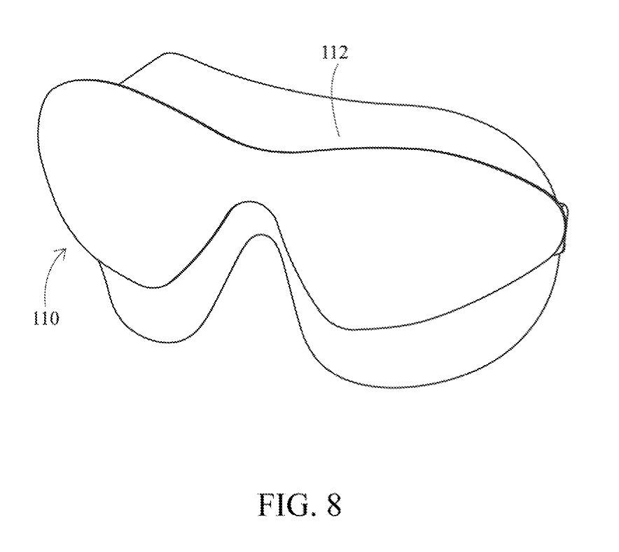

It shall be particularly appreciated that, in addition to the implementation as shown in FIG. 3 to FIG. 7 where two skirt portions 112 are respectively disposed on the left lens and the right lens, the skirt portion 112 of the lens structure 110 may also be formed to completely cover the lens structure 110 as shown in FIG. 8 and this can also achieve the waterproofing effect.

A second embodiment of a lens assembly of the present invention is described hereinafter.

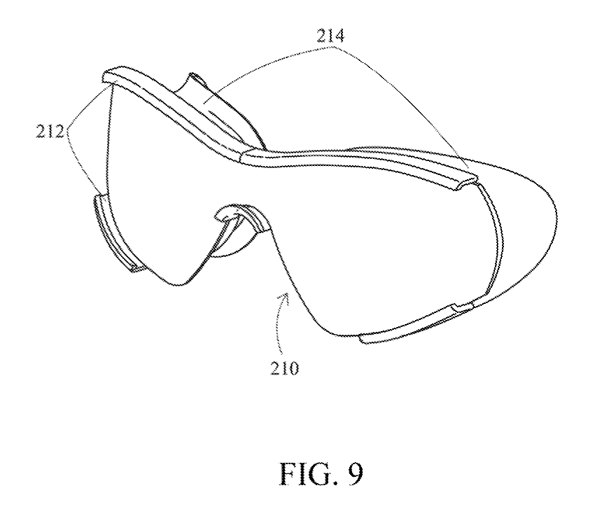

Referring to FIG. 9 to FIG. 12, in the second embodiment of the present invention, a lens assembly 200 comprises a first lens structure 210, a second lens structure 220, a fixing structure 230 and a quick release structure 240 so that the lens assembly 200 can be quickly released and assembled.

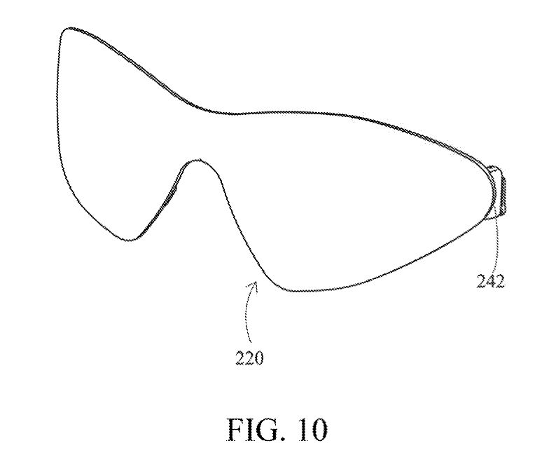

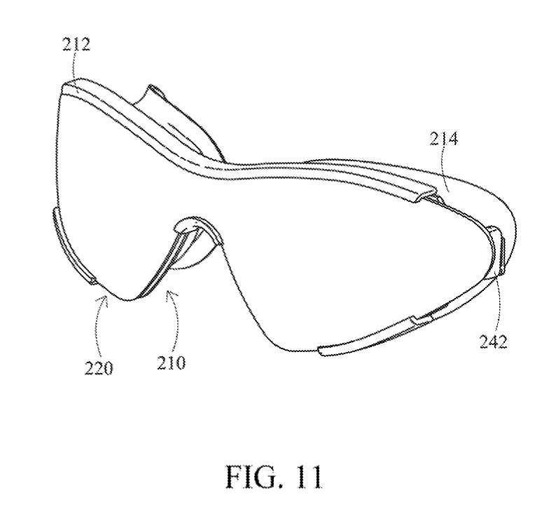

In detail, in the second embodiment, the first lens structure 210 is defined with an accommodating portion 212 at the front end thereof as shown in FIG. 9 so that the second lens structure 220 of FIG. 10 can be releasably disposed on the accommodating portion 212 of the first lens structure 210 to further form the assembled structure as shown in FIG. 11.

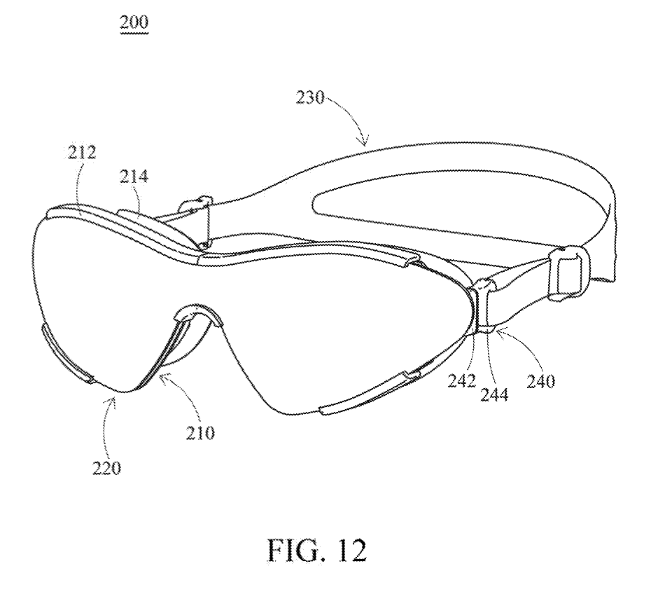

Similar to the first embodiment, the fixing structure 230 (which is a belt-type fixing structure in this case) is adapted to fix the first lens structure 210 around the eyes of a user (not shown) as shown in FIG. 12. The quick release structure 240 has a first quick release portion 242 and a second quick release portion 244, the first quick release portion 242 is disposed on the second lens structure 220, and the second quick release portion 244 is disposed on the fixing structure 230.

In this way, owing to the arrangement of the first quick release portion 242 and the second quick release portion 244, the second lens structure 220 and the fixing structure 230 can be rapidly assembled and disassembled by the user without using additional tools. On the other hand, the second lens structure 220 can also be rapidly disposed on the first lens structure 210 or disassembled from the first lens structure 210 without additional tools.

Different from the first embodiment, the first quick release portion 242 of the lens assembly 200 of the second embodiment is disposed on the second lens structure 220 by being assembled to or integrally formed with the second lens structure 220 so that it can be rapidly assembled with or disassembled from the fixing structure 230 that has the second quick release portion 244 disposed thereon.

Therefore, if the lens assembly 200 of the second embodiment of the present invention is designed having both the function of the goggles and that of the sunglasses, then the first lens structure 210 may be a pair of goggles and the second lens structure 220 may be a pair of sunglasses, and the first lens structure 210 may further comprise a skirt portion 214 so that the skirt portion 214 can cover the face of the user when the user is wearing the lens assembly 200 to achieve the waterproofing effect of the goggles.

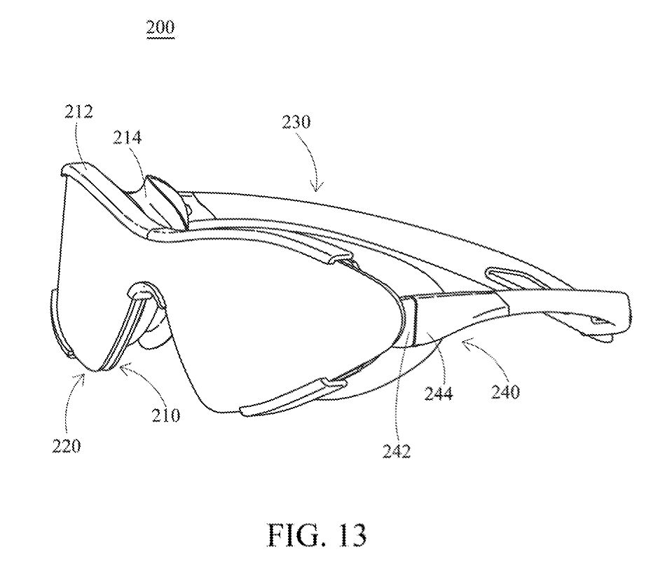

Of course, as shown in FIG. 13, the fixing structure 230 may also be selected to be a temple-type fixing structure by the user depending on his/her personal preference so that the lens assembly 200 is more comfortable to wear.

Furthermore, the quick release structure 240 is implemented in the same way as the first embodiment and may be a snap structure or a latch structure, and thus will not be further described herein.

A third embodiment of a lens assembly of the present invention is described hereinafter.

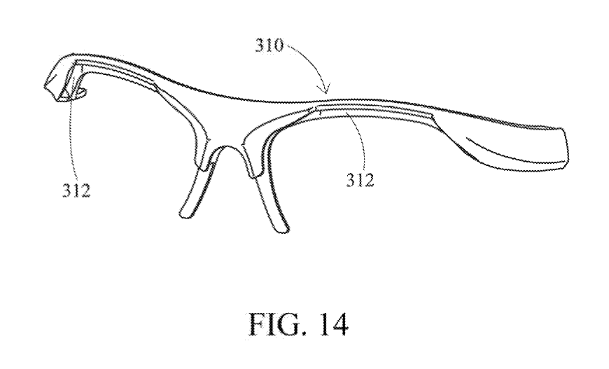

Referring to FIG. 14 to FIG. 17, when the lens assembly is to provide the sun shading effect in a first aspect of the third embodiment of the present invention, a lens assembly 300 comprises a lens frame 310, a set of lenses 320, a fixing structure 330 and a quick release structure 340, and the set of lenses 320 is a pair of sunglasses, and thereby the lens assembly 300 can be quickly released and assembled.

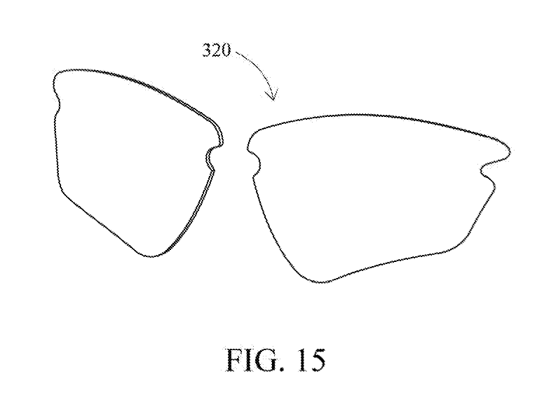

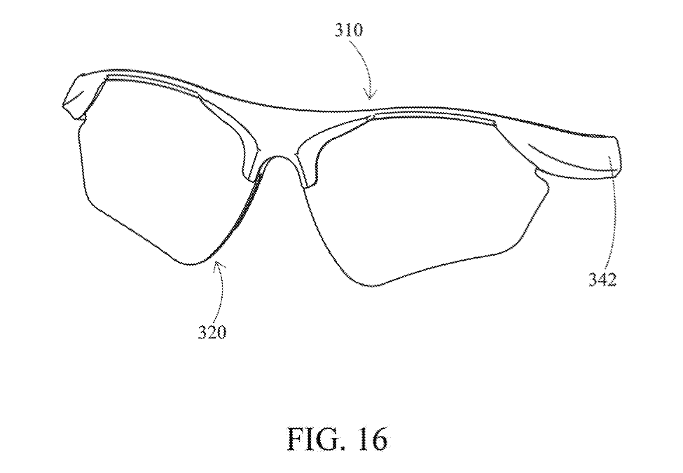

In detail, in the third embodiment, the lens frame 310 is only designed with an upper frame and defined with a groove 312 for accommodating the set of lenses 320 as shown in FIG. 14, so the set of lenses 320 corresponding to the lens frame 310 can thus be releasably clamped within the groove 312 of the lens frame 310 as shown in FIG. 15 to form the structure as shown in FIG. 16.

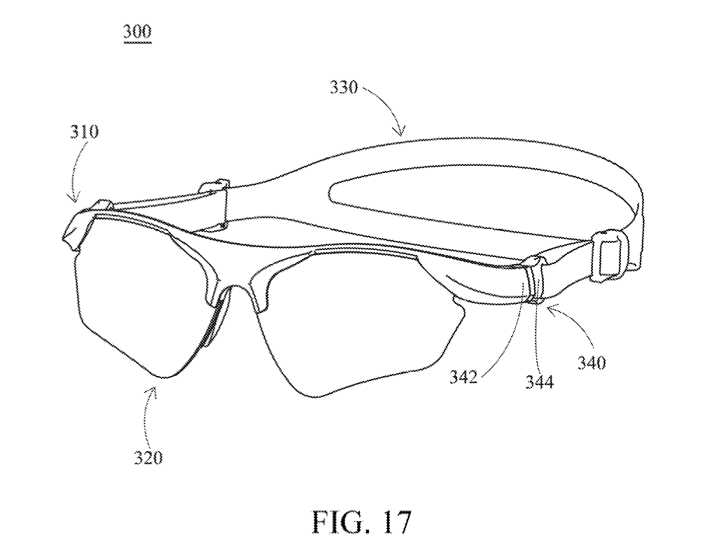

Similar to the aforesaid embodiments, the fixing structure 330 is adapted to fix the lens frame 310 around the eyes of a user (not shown) as shown in FIG. 17, and the quick release structure 340 has a first quick release portion 342 and a second quick release portion 344. The first quick release portion 342 is disposed on the lens frame 310, and the second quick release portion 344 is disposed on the fixing structure 330.

That is, owing to the arrangement of the first quick release portion 342 and the second quick release portion 344, the set of lenses 320 and the fixing structure 330 can be rapidly assembled or disassembled by the user without using additional tools. On the other hand, the set of lenses 320 can also be rapidly disposed on the lens frame 310 or disassembled from the lens frame 310 without additional tools.

Different from the first embodiment, the first quick release portion 342 of the lens assembly 300 of the third embodiment is disposed on the lens frame 310 by being assembled to or integrally formed with the lens frame 310 so that it can be rapidly assembled with or disassembled from the fixing structure 330 that has the second quick release portion 344 disposed thereon.

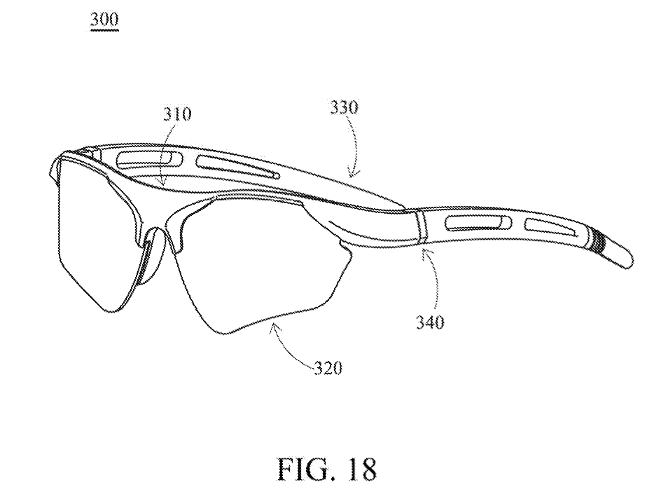

As shown in FIG. 18, the fixing structure 330 may also be selected to be a temple-type fixing structure by the user depending on his/her personal preference so that the lens assembly 300 is more comfortable to wear.

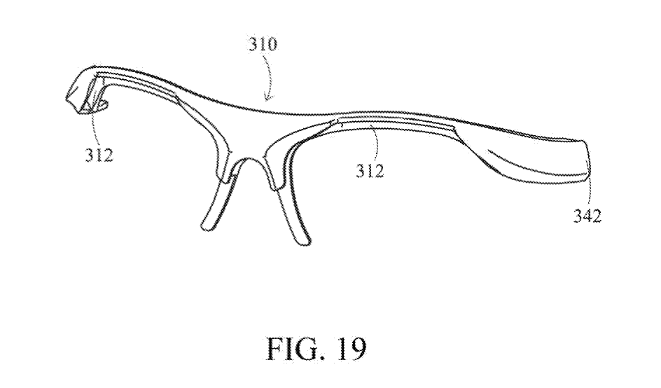

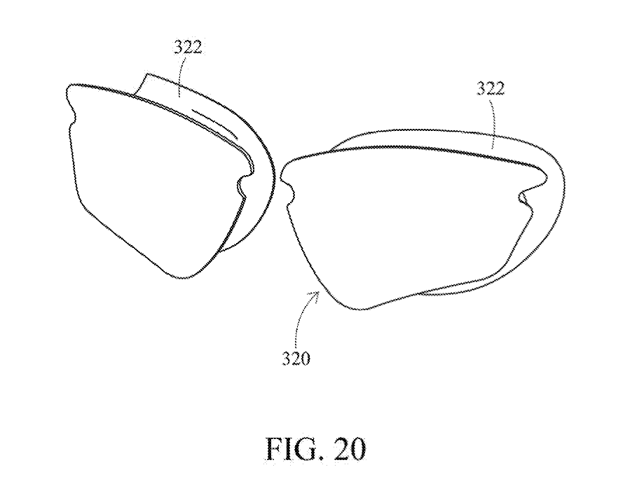

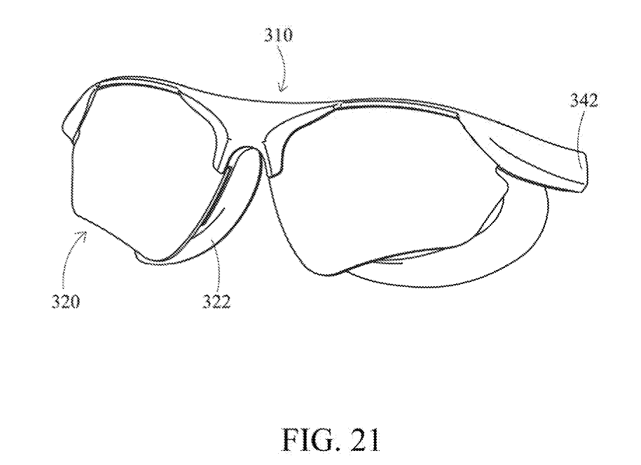

Additionally, when the lens assembly 300 is to be used as a pair of goggles in a second aspect of the third embodiment of the present invention, two lenses 322 of the set of lenses 320 may be respectively provided with a skirt portion 324 as shown in FIG. 20 in addition to the lens frame 310 as shown in FIG. 19. Thus, when the lens frame 310 is assembled with the set of lenses 320 as shown in FIG. 21, the skirt portion 324 can cover the face of the user when the user is wearing the lens assembly to achieve the waterproofing effect by using the set of lenses 320.

In detail, reference may be made to the following description for the arrangement of the skirt portion 324: first, each lens 322 defines a peripheral portion and an indentation portion at a place nearby but not contacting the peripheral portion, and then the skirt portion 324 is disposed on the indentation portion, thereby the arrangement of the skirt portion 324 is finished.

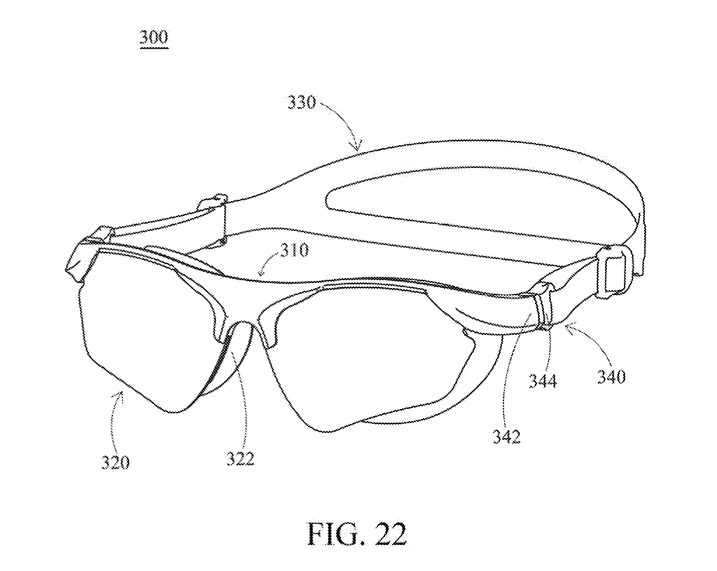

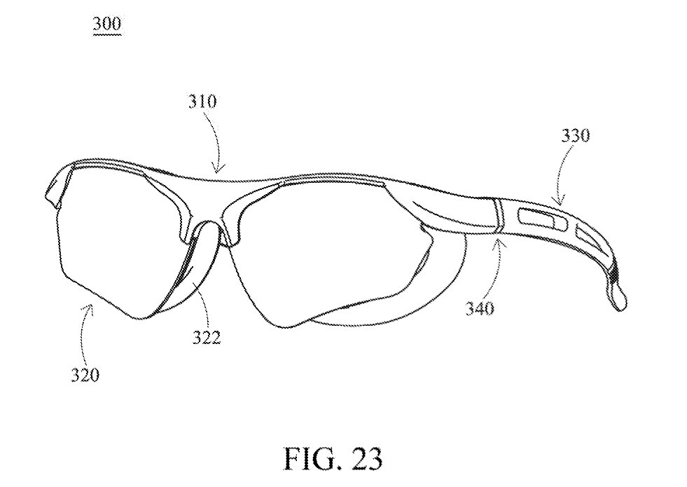

Of course, as shown in FIG. 22 and FIG. 23, the fixing structure 330 may also be selected to be a belt-type fixing structure or a temple-type fixing structure by the user depending on his/her personal preference so that the lens assembly 300 can be worn in different ways.

In this way, in the third embodiment of the present invention, by means of the arrangement of the groove 312 of the lens frame 310, the user can rapidly convert the set of lenses 320 into goggles or sunglasses depending on different usage environments and without using additional tools.

Additionally, owing to the arrangement of the first quick release portion 342 and the second quick release portion 344, the user can also rapidly convert the fixing structure into a temple-type fixing structure or a belt-type fixing structure without using additional tools.

According to the above descriptions, by virtue of the assembling relationships between the first quick release portion 132 and the second quick release portion 134 in the first embodiment, the assembling relationships between the first lens structure 210 and the second lens structure 220 in the second embodiment, and the arrangement of the groove 312 of the set of lenses 320 in the third embodiment of the present invention, the user in the triathlon sport or other activities can convert the lens assembly into goggles or sunglasses and rapidly replace the belt-type fixing structure with the temple-type fixing structure or vice versa depending on different usage environments and without using additional tools. Thus, the different wearing requirements are satisfied and the burden for the user to carry the goggles and the sunglasses is reduced.

The above disclosure is related to the detailed technical contents and inventive features thereof. People skilled in this field may proceed with a variety of modifications and replacements based on the disclosures and suggestions of the invention as described without departing from the characteristics thereof. Nevertheless, although such modifications and replacements are not fully disclosed in the above descriptions, they have substantially been covered in the following claims as appended.

What is claimed is:

1. A lens assembly that can be quickly released and assembled, comprising:
a lens structure;
a fixing structure, adapted to fix the lens structure around the eyes of a user; and
a quick release structure having a first quick release portion and a second quick release portion, the first quick release portion is disposed on the lens structure, and the second quick release portion is disposed on the fixing structure;
wherein the lens structure and the fixing structure can be assembled and disassembled rapidly by means of the first quick release portion and the second quick release portion without additional tools.

2. The lens assembly as claimed in claim 1, wherein the lens structure comprises a skirt portion that can cover the face of the user as wearing the lens structure.

3. The lens assembly as claimed in claim 1, wherein the lens structure is a pair of goggles or a pair of sunglasses, and the fixing structure is a temple-type fixing structure.

4. The lens assembly as claimed in claim 1, wherein the lens structure is a pair of goggles or a pair of sunglasses, and the fixing structure is a belt-type fixing structure.

5. The lens assembly as claimed in claim 1, wherein the quick release structure is a snap structure or a latch structure.

6. The lens assembly as claimed in claim 1, wherein the first quick release portion is disposed on the lens structure by being assembled to or integrally formed with the lens structure.

7. A lens assembly that can be quickly released and assembled, comprising:
a first lens structure;
a second lens structure being releasably disposed on the first lens structure;
a fixing structure adapted to fix the first lens structure around the eyes of a user; and
a quick release structure having a first quick release portion and a second quick release portion, the first quick release portion is disposed on the second lens structure and the second quick release portion is disposed on the fixing structure;
wherein the second lens structure and the fixing structure can be assembled and disassembled rapidly by means of the first quick release portion and the second quick release portion without additional tools.

8. The lens assembly as claimed in claim 7, wherein the second lens structure can be rapidly disposed on the first lens structure or disassembled from the first lens structure without additional tools.

9. The lens assembly as claimed in claim 7, wherein the first lens structure comprises a skirt portion that can cover the face of the user as wearing the lens structure.

10. The lens assembly as claimed in claim 7, wherein the first lens structure is a pair of goggles, the second lens structure is a pair of sunglasses, and the quick release structure is a snap structure or a latch structure.

11. The lens assembly as claimed in claim 7, wherein the first quick release portion is disposed on the second lens structure by being assembled to or integrally formed with the second lens structure.

12. A lens assembly that can be quickly released and assembled, comprising:
a lens frame;
a set of lenses adapted to be fixed on the lens frame;
a fixing structure adapted to fix the lens frame around the eyes of a user; and
a quick release structure, having a first quick release portion and a second quick release portion, the first quick release portion is disposed on the lens frame, and the second quick release portion is disposed on the fixing structure;
wherein the lens frame and the fixing structure can be assembled and disassembled rapidly by means of the first quick release portion and the second quick release portion without additional tools.

13. The lens assembly as claimed in claim 12, wherein the set of lenses is a pair of goggles or a pair of sunglasses.

14. The lens assembly as claimed in claim 13, wherein the set of lenses includes two lenses, each of the lenses has a peripheral portion, each of the lenses defines an indentation portion at a place nearby but not contacting the peripheral portion, and a skirt portion is disposed on the indentation portion.

15. The lens assembly as claimed in claim 12, wherein the fixing structure is a temple-type fixing structure or a belt-type fixing structure, and the quick release structure is a snap structure or a latch structure.

16. The lens assembly as claimed in claim 12, wherein the first quick release portion is disposed on the lens frame by being assembled to or integrally formed with the lens frame.
